(19) United States
(12) Patent Application Publication
(10) Pub. No.: US 2011/0015410 A1
(43) Pub. Date: Jan. 20, 2011

(54) NOVEL USE OF SCOPARONE

(75) Inventor: In Kyu Lee, Daegu-si (KR)

Correspondence Address: Jae Y. Park, Kile, Park, Goekjian, Reed & McManus, PLLC, 1200 New Hampshire Ave. NW, Suite 570, Washington, DC, 20036, US

(73) Assignee: Kyungpook National University Industry Academic Cooperation Foundation, Daegu-si (KR)

(21) Appl. No.: 12/677,092

(22) PCT Filed: Sep. 10, 2008
(86) PCT No.: PCT/KR2008/005324

§ 371 Date: Mar. 8, 2010

(30) Foreign Application Priority Data
Sep. 13, 2007 (KR) 10-2007-0093310

(51) Int. Cl. C07D 311/16

(52) U.S. Cl. 549/289

(57) ABSTRACT

Disclosed herein are a pharmaceutical composition for inhibiting the proliferation of vascular smooth muscle cells, which contains scoparone as an active ingredient, the use of scoparone for inhibiting the proliferation of vascular smooth muscle cells and a method for inhibiting the proliferation of vascular smooth muscle cells using scoparone. According to the disclosed invention, it has been found that scoparone can inhibit the proliferation of vascular smooth muscle cells by increasing the activity of AMPK. Accordingly, scoparone can be advantageously used as an active ingredient' in drugs for inhibiting the proliferation of vascular smooth muscle cells, particularly preventing or treating blood vessel restenosis.

### TECHNICAL FIELD

The present invention relates to a pharmaceutical composition for inhibiting the proliferation of vascular smooth muscle cells, which contains scoparone as an active ingredient, the use of scoparone for inhibiting the proliferation of vascular smooth muscle cells and a method for inhibiting the proliferation of vascular smooth muscle cells using scoparone.

### BACKGROUND ART

The proliferation of vascular smooth muscle cells is an important cause of arteriosclerosis including atherosclerosis, and cardiovascular diseases including blood vessel restenosis (Hidde B., Restenosis: a challenge for pharmacology. Trends. Pharmacol. Sci. 2000; 21(7):274-279; Nageswara R M, and Marschall S R, Circ. Res. 2007; 100:460-473: Andres V, Castro C. Antiproliferative strategies for the treatment of vascular proliferative disease. Curr Vasc Pharmacol. 2003 March; 1(1):85-98: Hao H, Gabbiani G, Bochaton-Piallat M L. Arterial smooth muscle cell heterogeneity: implications for atherosclerosis and restenosis development. Arterioscler Thromb Vasc Biol. 2003 Sep. 1; 23(9):1510-20).

The best way to prevent such cardiovascular diseases is to control factors such as hypertension, hyperlipidemia, obesity and diabetes. However, if such diseases develop, treatments that use drugs or surgical methods are required. Blood pressure is controlled using statin-based drugs and antihypertensive drugs, but this blood pressure control cannot provide fundamental treatment, because it reduces cardiovascular diseases only by about 15-30%. The best treatment method known to date is to open blood vessels by inserting a balloon catheter into blood vessels which have clogged or become narrow, and then dilating the balloon (Hidde B., Restenosis: a challenge for pharmacology. Trends. Pharmacol. Sci. 2000; 21(7):274-279). However, there occurs a problem in that a restenosis rate of about 50% is shown within about 1 year after balloon dilatation due to the reproliferation of vascular smooth muscle cells. For this reason, it is necessary to inhibit the proliferation of vascular smooth muscle cells.

Recently, studies on the relationship between various metabolic diseases and mitochondria have been actively conducted. In the pathogenic mechanisms of vascular complications, it was observed that oxidative stress in vascular cells increased. It is generally known that this increase in oxidative stress is attributable to the dysfunction of mitochondria (Nageswara R M and Marschall S R, Circ. Res. 2007; 100:460-473). This is because mitochondria are organelles that produce reactive oxygen species within vascular cells on glucose metabolism and lipid metabolism among various oxidative stress-generating systems and can also commonly act on oxidative stress caused by high blood glucose levels, fatty acids, cytokines and growth factors to accelerate the development of vascular complications. In recent studies, it was observed that the overexpression of genes such as UCP-2, AMPK and PGC-1 improved the function of mitochondria by hypertension inducers and inhibited the proliferation and migration of vascular smooth muscle cells (Lee W. J., et al., Arterioscler Thromb Vasc Biol. 2005; 25:2488-2494; Park J. Y., et al., Diabetologia 2005; 48:1022-1028; Lee I K, et al., Effects of Recombinant Adenovirus-Mediated Uncoupling Protein 2 Overexpression on Endothelial Function and Apoptosis. Circ Res. 2005 Jun. 10; 96(11):1200-7; Kim H J, et al., Effects of PGC-1α on TNF-α Induced MCP-1 and VCAM-1 Expression and NF-κB Activation in Human Aortic Smooth Muscle and Endothelial Cells. ANTIOXIDANTS & REDOX SIGNALING. 2007; 9(3): 301-307).

It was reported again that the proliferation of vascular smooth muscle cells could be inhibited by the activity of AMPK (Nagata D, et al., AMP-activated protein kinase inhibits Angiotensin II-stimulated vascular smooth muscle cell proliferation. Circulation. 2004; 110:444-451). It was observed that the proliferation of vascular smooth muscle cells with activated AMPK was inhibited, and in such vascular smooth muscle cells, the expression of the cell proliferation inhibitors p53 and p21 increased and the activity of CDK (cyclin-dependent kinase) decreased (Igata M, et al., Adenosine monophosphate-activated protein kinase suppresses vascular smooth muscle cell proliferation through the inhibition of cell cycle progression. Circ Res. 2005; 97(8):837-844). AMPK is a kind of kinase which is activated when the relative ratio of AMP is higher than ATP by dietary restriction or exercise, and it is a metabolism-related important protein that functions to stop the replication of cells so as to inhibit further consumption of ATP (Hardie D G. AMP-activated protein kinase as a drug target. Annu. Rev. Pharmacol. Toxicol. 2007; 47:185-210). Activated AMPK is known to promote glucose metabolism and lipid oxidation and to inhibit gluconeogenesis and lipid synthesis. In addition, AMPK is also activated regardless of a metabolic process. Namely, it is also activated either by metformin known as a diabetes-treating agent or by alpha-lipoic acid (Lee W. J., et al., Arterioscler Thromb Vasc Biol. 2005; 25:2488-2494; Lee K M, et al., Alpha-lipoic acid inhibits fractalkine expression and prevents neointimal hyperplasia after balloon injury in rat carotid artery. Atherosclerosis. 2006 November; 189(1): 104-14).

Scoparone (6,7-dimethoxycoumarin) is a coumarin derivative that is a phenolic substance extracted from plants, and it is constituted by a benzene ring and an α-pyrone ring fused together. Coumarins are components extracted from Artemisia scoparia, Artemisia capillaris, Artemisia princes and the like and are used as agents for treating or alleviating various diseases. Among them, scoparone is mainly extracted from Artemisia scoparia and has been reported to have effects of immune suppression, vascular relaxation, lipid lowering, etc. Scoparone also inhibits the growth of human peripheral monocytes, and it was observed in a high-cholesterol rabbit model that scoparone lowered triglyceride and cholesterol levels. Moreover, scoparone has been reported to have positive effects on asthma. In addition, scoparone has been reported to have various pharmacological actions, including blood pressure-lowering action, choleretic action, anti-inflammatory action, etc. Furthermore, Taiwanese Huang et al. found that scoparone showed blood vessel-relaxing action and immune-suppressing action.

The present inventors have studied substances promoting the activity of AMPK in vascular smooth muscle cells and, as a result, have found that scoparone inhibits the proliferation of vascular smooth muscle cells by promoting the activity of AMPK in vascular smooth muscle cells, thereby completing the present invention.

### DISCLOSURE

### Technical Problem

Therefore, it is an object of the present invention to provide a pharmaceutical composition for inhibiting the proliferation of vascular smooth muscle cells, which contains scoparone as an active ingredient, the use of scoparone for inhibiting the proliferation of vascular smooth muscle cells, and a method for inhibiting the proliferation of vascular smooth muscle cells using scoparone.

### Technical Solution

In one aspect, the present invention provides a pharmaceutical composition for inhibiting the proliferation of vascular smooth muscle cells, which contains scoparone as an active ingredient.

According to the present invention, it has been found that scoparone inhibits the proliferation of vascular smooth muscle cells and also reduces the formation of neointima which can be produced after balloon dilatation. As can be seen in Examples below, scoparone inhibits the proliferation of vascular smooth muscle cells by activating AMPK and induces the activation of AMPK and the inhibition of phosphorylation/activity of ACC2 by influencing the upstream signaling network of AMPK. Furthermore, scoparone increases the expression of the cell cycle inhibitory proteins p21, p27 and p53 and reduces the expression of the cell cycle regulatory protein cyclin D. In addition, scoparone reduces the production of ROS in blood vessels and also dose-dependently reduces the expression of VCAM-1 protein, the expression of which is increased with the increase of ROS.

As described above, it has been found that scoparone inhibits the proliferation of vascular smooth muscle cells through the activation of AMPK. Accordingly, scoparone can be used as an active ingredient in a drug for inhibiting the proliferation of vascular smooth muscle cells.

The inventive composition containing scoparone as an active ingredient may comprise, in addition to the active ingredient, pharmaceutically suitable and physiologically acceptable adjuvants. Examples of the adjuvants include excipients, disintegrants, sweeteners, binders, coating agents, swelling agents, lubricants, flavoring agents, solubilizers, etc.

For administration, the inventive composition may also contain at least one pharmaceutically acceptable carrier, in addition to the active ingredients as described above.

The inventive composition containing scoparone as an active ingredient may be formulated in the form of granules, powders, tablets, coated tablets, capsules, suppositories, syrup, juice, suspensions, emulsions, or injectable liquids.

For instance, for formulation in the form of tablets or capsules, the active ingredient may be combined with any oral nontoxic pharmaceutically acceptable inert carrier such as ethanol, glycerol or water. Moreover, when desired or necessary, suitable binders, lubricants, disintegrating agents and coloring agents can also be incorporated into the mixture. Suitable binders include, but are not limited to, starch, gelatin, natural sugars such as glucose or beta-lactose, corn sweeteners, natural and synthetic gums such as gum acacia, tragacanth gum or sodium oleate, sodium stearate, magnesium stearate, sodium benzoate, sodium acetate, sodium chloride, etc. Suitable disintegrants include, but are not limited to, starch, methyl cellulose, agar, bentonite, xanthan gum, etc.

Examples of pharmaceutically acceptable carriers, which can be used to formulate the inventive composition in the form of liquid solutions, include saline solution, sterile water, Ringer's solution, buffered saline solution, dextrose solution, maltodextrin solution, glycerol, ethanol, and a mixture of two or more thereof. If necessary, the inventive composition may also contain other conventional additives, such as antioxidants, buffers and bacteriostatic agents. Moreover, the inventive composition may additionally contain diluents, dispersants, surfactants, binders and lubricants in order to formulate it into injection formulations, such as aqueous solutions, suspensions and emulsions, pills, capsules, granules and tablets. Furthermore, the inventive composition may preferably be formulated depending on particular diseases and its components, using the method described in Remington's Pharmaceutical Science, Mack Publishing Company, Easton Pa., which is a suitable method in the relevant field of art.

In another aspect, the present invention provides the use of scoparone for preparing drugs for inhibiting the proliferation of vascular smooth muscle cells.

The pharmaceutical composition for inhibiting the proliferation of vascular smooth muscle cells can be used to prepare such drugs.

In still another aspect, the present invention provides a method for inhibiting the proliferation of vascular smooth muscle cells, which comprises administering to mammals a pharmaceutical composition containing a therapeutically effective amount of scoparone as an active ingredient.

In the present invention, the inhibition of the proliferation of vascular smooth muscle cells includes reducing and preventing the proliferation of vascular smooth muscle cells.

The inventive pharmaceutical composition for inhibiting the proliferation of vascular smooth muscle cells can be used for the prevention or treatment of arteriosclerosis including atherosclerosis, and cardiovascular diseases including blood vessel restenosis, which are caused by the proliferation of vascular smooth muscle cells (Hidde B., Restenosis: a challenge for pharmacology. Trends. Pharmacol. Sci. 2000; 21(7):274-279; Nageswara R M, and Marschall S R, Circ. Res. 2007; 100:460-4; Andres V, Castro C. Antiproliferative strategies for the treatment of vascular proliferative disease. Curr Vase Pharmacol. 2003 March; 1(1):85-98; Hao H, Gabbiani G, Bochaton-Piallat M L. Arterial smooth muscle cell heterogeneity: implications for atherosclerosis and restenosis development. Arterioscler Thromb Vase Biol. 2003 Sep. 1; 23(9):1510-20).

Accordingly, the inventive composition for inhibiting the proliferation of vascular smooth muscle cells may also contain one or more agents for treating cardiovascular diseases. For example, scoparone may be used in combination with a hyperlipidemia therapeutic agent or a blood pressure-lowering agent, which are well known to those skilled in the art.

The inventive composition containing scoparone as an active ingredient may be administered in the conventional manner via the subcutaneous, intravenous, intraarterial, intraabdominal, intramusclar, intrasternal, percutaneous, intranasal, inhalation, topical, rectal, oral, intraocular or intradermal route.

“The therapeutically effective amount” of the inventive composition containing scoparone as an active ingredient refers to the amount needed to achieve the effect of inhibiting the proliferation of vascular smooth muscle cells. Accordingly, the therapeutically effective amount may vary depending on various factors, including the kind and severity of diseases, the kind and content of an active ingredient and other components contained in the composition, the kind of a formulation, the patient's age, weight, general health condition, sex and diet, administration time, administration route, the secretion ratio of the composition, administration period, and the kind of drugs used in combination. Scoparone is preferably administered at a dose of 10-1000 mg/kg once or several times a day for adults.

### Advantageous Effects

According to the present invention, it has been found that scoparone can inhibit the proliferation of vascular smooth muscle cells by increasing the activity of AMPK. Accordingly, scoparone can be advantageously used as an active ingredient in drugs for inhibiting the proliferation of vascular smooth muscle cells, particularly preventing or treating blood vessel restenosis.

### DESCRIPTION OF DRAWINGS

FIG. 1 is a graphic diagram showing that the proliferation of vascular smooth muscle cells was significantly decreased in a manner dependent on the concentration of scoparone, when the cells were treated with scoparone along with PDGF or TNF-α.

FIG. 2 is a micrograph (×100) showing the cross-section of the carotid artery of rats 2 weeks after balloon dilatation.

FIG. 3 is a Western blot photograph showing the effect of scoparone on the phosphorylation of AMPK and ACC.

FIG. 4 is a Western blot photograph showing the effect of scoparone on the expression of the cell proliferation-related proteins p53, p21, p27 and cyclin D.

FIG. 5 is a Western blot photograph showing the effect of scoparone on the phosphorylation of JNK and Erk.

FIG. 6 is, a fluorescence microscope showing the effect of scoparone on the inhibition of the production of ROS.

FIG. 7 is a Western blot photograph showing the effect of scoparone on the expression of VCAM-1 protein.

FIG. 8 shows electrophoretic mobility shift assay results indicating the effects of scoparone on the DNA-binding activities of AP-1 and NF-κB.

### BEST MODE

The advantages and features of the present invention and methods for achieving them will become more apparent from the following examples. However, the present invention is not limited to the illustrated examples and may be embodied in various different forms. Rather, these examples are provided so that the disclosure of the present invention will be thorough and complete, and will fully convey the scope of the invention to those skilled in the art to which the present invention pertains. The scope of the present invention will only be defined by the appended claims.

### Examples

Isolation and culture of Vascular Smooth Muscle Cells

Vascular smooth muscle cells were isolated from the thoracic aorta of Sprague-Dawley white rats and cultured in a medium containing 20% fetal bovine serum.

The specificity of vascular smooth muscle cells was confirmed by staining the cells with α-actin monoclonal antibody (Sigma, St Louis, Mo., USA). In this experiment, vascular smooth muscle cells subcultured 5-6 times were used. The cultured vascular smooth muscle cells were plated in a 60-mm tissue culture dish at a confluence of about 80-90%, and then cultured in 0.5% FBS DMEM medium for 24 hours to allow the cells to enter the stationary phase.

### Example 1

### Analysis of Effect of Scoparone on Inhibition of Proliferation of Vascular Smooth Muscle Cells

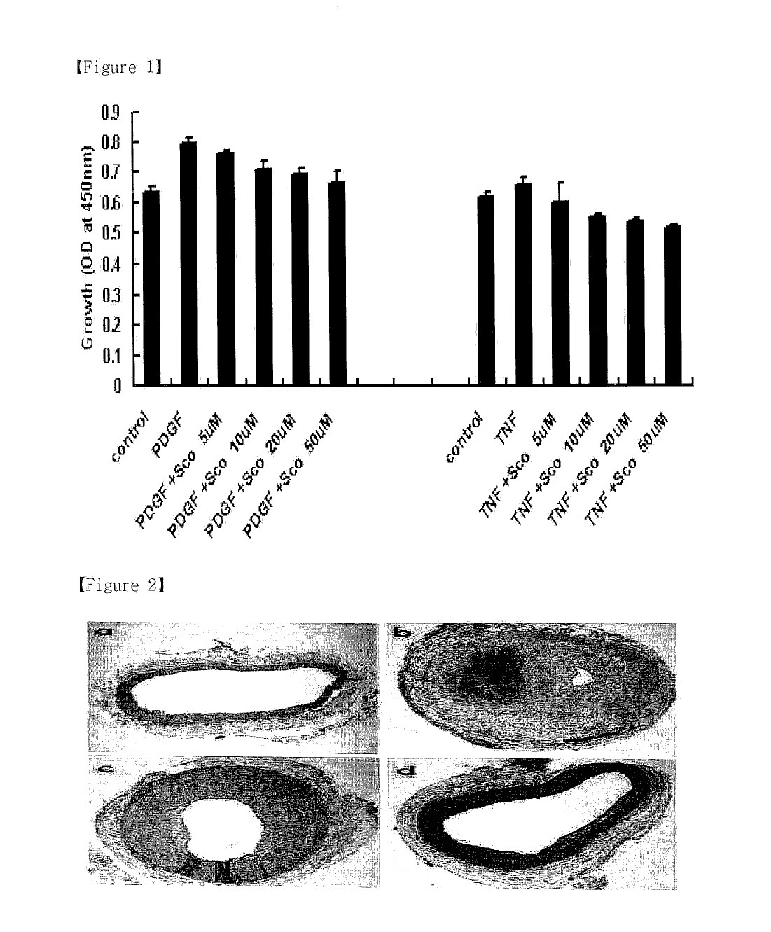

The primarily cultured vascular smooth muscle cells were cultured in a 96-well culture dish, and when the cells reached a confluence of 40%, the medium was replaced with 0.5% FBS-containing medium, and the cells were cultured for 24 hours to allow the cells to enter the stationary phase. Then, the cells were treated with 0, 5, 10, 20 or 50 μM of scoparone along with 20 ng/ml of platelet-derived growth factor (PDGF) or 10 ng/ml of tumor necrosis factor (TNF-α and incubated at 37° C. for 48 hours. The number of the cells was counted with a WST cell counting kit (WAKO, Japan). After the cells were treated with a proliferation reagent (WST), the cells were further incubated for 4 hours, and the absorbance at 450 nm was measured with an ELISA reader to determine the proliferation capacity of the cells. As can be seen in FIG. 1, when the cells were treated with platelet-derived growth factor (PDGF) or TNF-α the proliferation of the vascular smooth muscle cells was increased, but when the cells were treated with platelet-derived growth factor (PDGF) or TNF-α along with scoparone, the proliferation of the vascular smooth muscle cells was decreased in a dose-dependent manner.

### Mode for Invention

### Example 2

### Examination of Effect of Inhibiting Proliferation of Vascular Smooth Muscle Cells in Sprague-Dawley White Rats

In order to examine whether scoparone inhibits the formation of neointima after balloon dilatation, an experiment was performed using Sprague-Dawley white rats fed with scoparone-containing feed.

As test subjects, male Sprague-Dawley white rats weighing about 300 g were used. The rats were grouped into a normal control group, a negative control group fed only with a high-fat diet (20% fat and 0.05% cholesterol) and a test group fed with a high-fat diet containing 10 mg/kg or 100 mg/kg of scoparone, each group consisting of 4 animals, and were kept at 22° C. under a 12-hr light/12-hr dark cycle. The negative control group and the test group were fed with the above-described diets from 3 days before performing balloon dilatation and were fed with the diets during 2 weeks after balloon dilatation. After 2 weeks, the carotid artery was isolated from the rats and stained with H&E (hematoxylin & eosin) in order to observe the formation of neointima. FIG. 2 is a micrograph (×100) showing the cross-section of the carotid artery of the rats 2 weeks after balloon dilatation. Specifically, FIG. 2a shows the results of H&E staining for the normal control group, FIG. 2b shows the results of H&E staining for the negative control group, FIG. 2c shows the results of H&E staining for the group fed with 10 mg/kg of scoparone, and FIG. 2d shows the results of H&E staining for the group fed with 100 mg/kg of scoparone. As can be seen in FIG. 2, the formation of neointima in the groups fed with scoparone was decreased compared to that in the negative control group, and the decrease rate of neointima formation was increased with an increase in the dose of scoparone. From the above test results, it can be found that scoparone can prevent or treat blood vessel restenosis after balloon dilatation by inhibiting the proliferation of vascular smooth muscle cells.

### Test Example 1

### Analysis of Effect of Scoparone on Phosphorylation of AMPK and ACC

Cultured vascular smooth muscle cells were plated in a 60-mm tissue culture dish at a confluence of about 80-90%, and then cultured in 0.5% FBS-containing medium for 24 hours to allow the cells to enter the stationary phase. The cultured cells were divided into a control group not treated with scoparone, and five test groups which were treated with 50 μg of scoparone for 1 hr, 2 hr, 4 hr, 6 hr and 12 hr, respectively. From each of the groups, the total protein was isolated using RIPA buffer. Each of the isolated total proteins was boiled in buffer for 5 minutes, and then cooled on ice. Then, each total protein was separated according to size by electrophoresis on sodium dodecyl sulfate polyacrylamide. Then, each total protein was transferred to a PVDF membrane which was then allowed to react with monoclonal antibodies for pACC, pAMPK and AMPK to examine the expression and phosphorylation of the proteins.

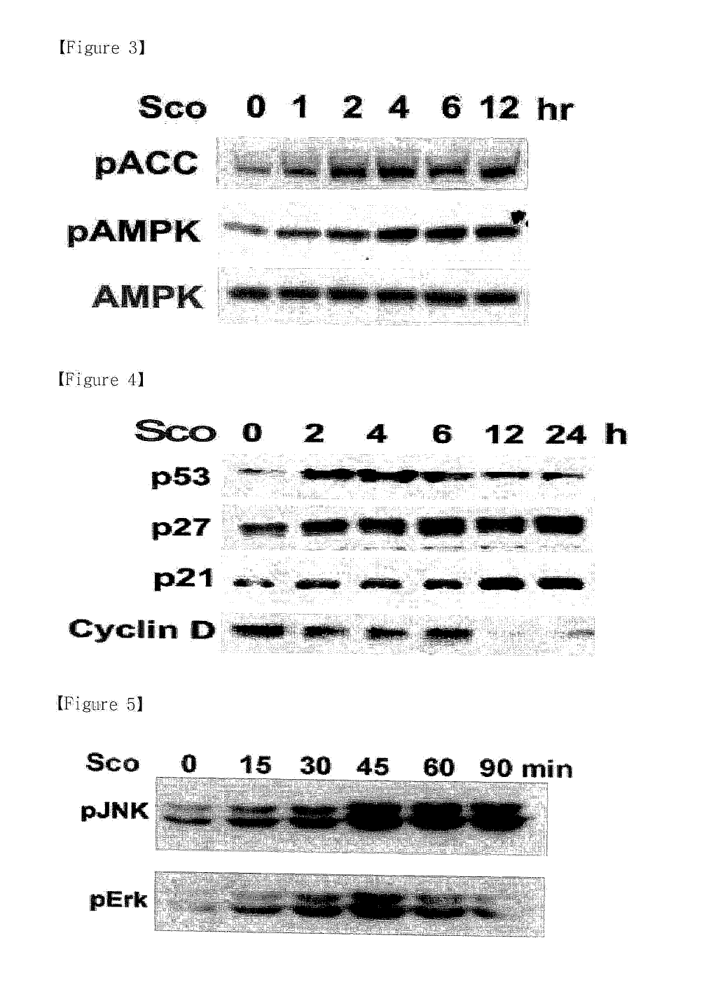

As can be seen in FIG. 3, when the vascular smooth muscle cells were treated with scoparone, the phosphorylation of AMPK and the resulting phosphorylation of ACC was time-dependently increased.

### Test Example 2

### Analysis of Effect of Scoparone on Expression of Cell Proliferation-Related Proteins

Cultured vascular muscle cells were plated in a 60-mm tissue culture dish at a confluence of about 80-90%, and then cultured in 0.5% FBS containing medium for 24 hours to allow the cells to enter the stationary phase. The cultured cells were divided into a control group not treated with scoparone, and five test groups which were treated with 50 μg of scoparone for 2 hr, 4 hr, 6 hr, 12 hr and 24 hr, respectively. From each of the groups, the total protein was isolated using RIPA buffer. Each of the isolated total proteins was boiled in buffer for 5 minutes, and then cooled on ice. Then, each total protein was separated according to size by electrophoresis on sodium dodecyl sulfate polyacrylamide gel. Then, each total protein was transferred to a PVDF membrane which was then allowed to react with antibodies for p53, p27, p21 and Cyclin D to examine the expression of the proteins.

As can be seen in FIG. 4, when the vascular smooth muscle cells were treated with scoparone, the expression of the cell cycle-related proteins p53, p27 and p21 was time-dependently increased. It could be observed that the expression levels of the cell cycle inhibitory proteins p21 and p27 were increased with the passage of time after treatment with scoparone and were the highest after 24 hours. Also, p53 showed the highest expression level at 2-4 hours after treatment with scoparone. The expression level of the cell cycle regulatory protein cyclin D was decreased by treatment with scoparone.

### Test Example 3

### Analysis of Effect of Scoparone on Phosphorylation of JNK and Erk

In order to examine the signaling pathway of scoparone, the phosphorylation of JNK and Erk was examined.

Cultured vascular smooth muscle cells were plated in a 60-mm tissue culture dish at a confluence of 80-90%, and then cultured in 0.5% FBS-containing medium for 24 hours to allow the cells to enter the stationary phase. The cultured cells were divided into a control group not treated with scoparone, and five test groups which were treated with 50 μg of scoparone for 15 min, 30 min, 45 min, 60 min and 90 min, respectively. From each of the groups, the total protein was isolated using RIPA buffer. Each of the isolated total proteins was boiled in buffer for 5 minutes, and then cooled on ice. Then, each total protein was separated according to size by electrophoresis on sodium dodecyl sulfate polyacrylamide gel. Then, each total protein was transferred to a PVDF membrane which was then allowed to react with antibodies for pJNK, JNK, pErk and Erk to examine the expression and phosphorylation of the proteins.

As a result, it was observed that the phosphorylation of JEK was gradually increased with the passage of time. The phosphorylation of Erk was shown to be the highest at 45 min after treatment with scoparone. This suggests that JNK and Erk are involved in cell cycle regulation induced by scoparone.

FIG. 5 is a Western blot photograph showing the effect of scoparone on the phosphorylation of JNK and Erk.

### Test Example 4

### Analysis of Effect of Scoparone on ROS Production

When vascular smooth muscle cells were grown to a confluence of about 90% in a 6-well cell culture dish, the cells were cultured in 0.5% FBS DMEM medium for 24 hours. The cultured cells were divided into a control group treated with neither tumor necrosis factor (TNF-α nor scoparone, and three test groups which were treated with scoparone in tumor necrosis factor (TNF-α)-containing media at scoparone concentrations of 0 μM, 100 μM and 200 μM, respectively. The cells of each group were incubated for 1 hour, and then 40 μmol/L of 2′,7′-dichlorofluorecin diacetate (DCF-DA; Invitrogen), a fluorescent probe sensitive to ROS, was added thereto, and the cells were incubated for 30 minutes. The production of ROS in the cells was analyzed using an AxioCam MRc5 Carl Zeiss fluorescence microscope (Thornwood, N.Y.) which was excited at a 488-nm wavelength and emitted at 515-nm wavelength. As can be seen in FIG. 6, the expression of ROS was decreased in the groups treated with scoparone.

### Test Example 5

### Analysis of Effect of Scoparone on Expression of VCAM-1

Cultured vascular smooth muscle cells were plated in a 60-mm tissue culture dish at a confluence of about 80-90%, and then cultured in 0.5% FBS-containing medium for 24 hours to allow the cells to enter the stationary phase. The cultured cells were divided into a control group treated with neither scoparone nor tumor necrosis factor (TNF-α, and five test groups which were treated with scoparone in tumor necrosis factor (TNF-α)-containing media for 24 hours at scoparone concentrations of 0 μM, 10 μM, 20 μM, 50 μM and 100 μM, respectively. In addition, the cells were divided into five groups which were treated with 50 μg of scoparone for 15 min, 30 min, 45 min, 60 min and 90 min, respectively. From each of the groups, the total protein was isolated using RIPA buffer. Each of the isolated total protein was boiled in buffer for 5 minutes, and then cooled on ice. Then, each total protein was separated according to size by electrophoresis on sodium dodecyl sulfate polyacrylamide gel. Then, each total protein was transferred to a PVDF membrane which was then allowed to react with antibodies for VCAM and PAI-1 to examine the expression of the proteins. The membrane was further allowed to react with anti-actin antibody to examine whether the antibody uses a given amount of the proteins.

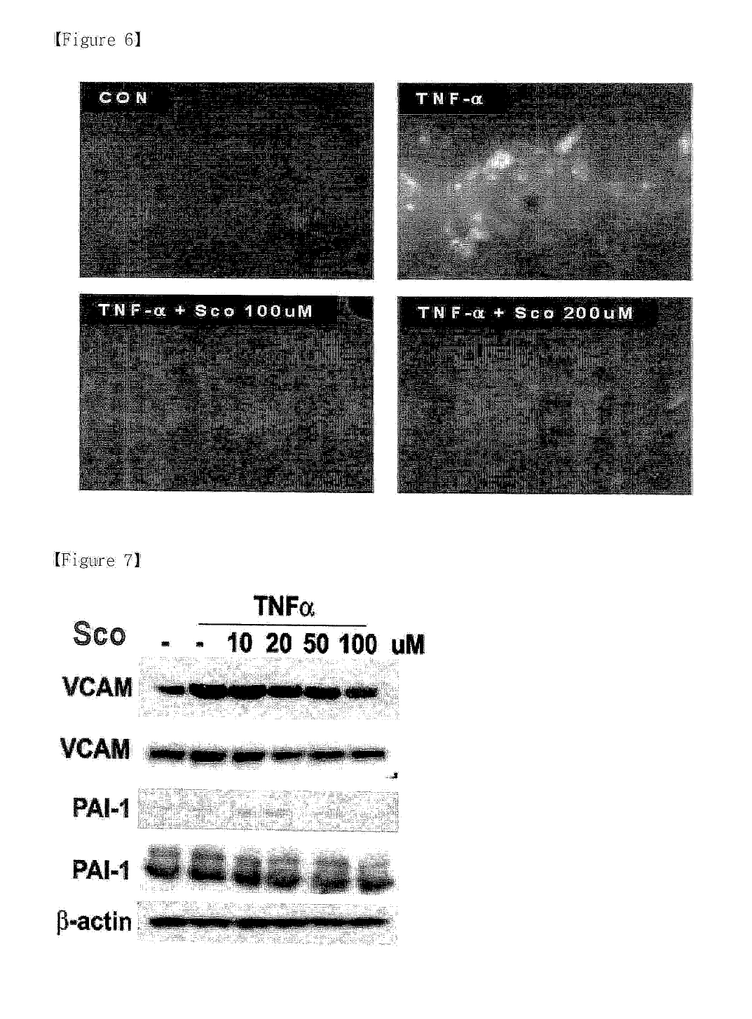

The increase of ROS leads to a remarkable increase in the expression of VCAM-1 protein that is a major cause of arteriosclerosis. As can be seen in FIG. 7, when the vascular smooth muscle cells were treated with scoparone, the expression of VCAM-1 was dose-dependently decreased.

### Test Example 6

### Analysis of Effect of Scoparone on DNA-Binding Activities of AP-1 and NFκB

Proteins such as cell cycle regulatory proteins or chemokine are regulated by the respective transcription factors. Accordingly, the DNA-binding activities of AP-1 that is a transcription factor regulating the expression of cell cycle regulatory proteins, and NF-κB that is a transcription factor regulating the expression of chemokine were analyzed using an electrophoretic mobility shift assay (EMSA).

Vascular smooth muscle cells were cultured in 0.5% FBS-containing medium for 24 hours. The cultured cells were divided into a control group treated with neither scoparone nor tumor necrosis factor (TNF-α, and five test groups which were treated with media containing 10 ng of tumor necrosis factor (TNF-α) for 24 hours at scoparone concentrations of 0 μM, 10 μM, 20 μM, 50 μM and 100 μM, respectively. Nuclear extracts were isolated from the vascular smooth muscle cells and labeled with radioisotope-labeled probes for AP-1 and NF-κB. Then, the labeled extracts were subjected to a protein-DNA reaction at room temperature for 20 minutes. After completion of the reaction, each sample was loaded on a 4% native polyarylamide gel and electrophoresed at 150 volt for 2 hours, followed by analysis.

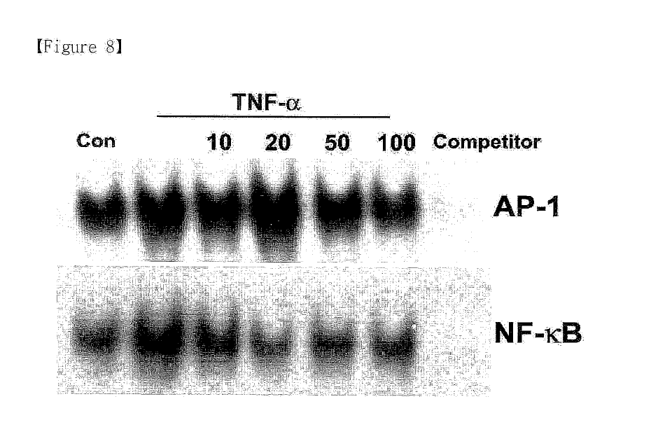

As a result, as can be seen in FIG. 8, the DNA binding activity of each transcription factor, which has been increased due to TNF-α was concentration-dependently decreased by treatment with scoparone. In addition, when the vascular smooth muscle cells were treated with CompC (Competitor, AMPK inhibitor, MERCK, Cat. #171260) that is an inhibitor of scoparone, the proliferation of the vascular smooth muscle cells was restored again.

### INDUSTRIAL APPLICABILITY

According to the present invention, it has been found that scoparone can inhibit the proliferation of vascular smooth muscle cells by increasing the activity of AMPK. Accordingly, scoparone can be advantageously used as an active ingredient in drugs for inhibiting the proliferation of vascular smooth muscle cells, particularly preventing or treating blood vessel restenosis.

1. A pharmaceutical composition for inhibiting the proliferation of vascular smooth muscle cells, which contains scoparone as an active ingredient.

2. The pharmaceutical composition of claim 1, wherein the pharmaceutical composition is used for the prevention or treatment of blood vessel restenosis.
